(19) United States
(12) Patent Application Publication
(10) Pub. No.: US 2024/0091505 A1
(43) Pub. Date: Mar. 21, 2024

(54) GUIDEWIRES AND METHODS OF DEPLOYMENT THEREOF

(71) Applicant: Covidien LP, Mansfield, MA (US)

(72) Inventor: Nathan J. Knutson, Long Lake, MN (US)

(21) Appl. No.: 18/275,802

(22) PCT Filed: Mar. 2, 2022
(86) PCT No.: PCT/US2022/018439

§ 371 Date: Aug. 3, 2023

Related U.S. Application Data
(60) Provisional application No. 63/155,956, filed on Mar. 3, 2021.

(51) Int. Cl. A61M 25/09

(52) U.S. Cl. CPC A61M 25/09; A61M 2025/09116

(57) ABSTRACT

The present disclosure describes guidewires including distal tips including enhanced cutting characteristics and methods of use associated therewith.

### BACKGROUND

### Technical Field

The present technology is generally related to guidewires, and more particularly, guidewires designed for accessing targets located beyond a defined luminal body structure and including enhanced cutting characteristics.

### Description of Related Art

A wide variety of guide wires have been developed. Of these known devices, each has certain advantages and disadvantages. However, there is an ongoing need to provide alternative guidewires and methods of using such guidewires. For example, in some instances, some known guidewires may display difficulty accurately accessing a target residing off-lumen or outside a given bodily lumen. Particularly, guidewires configured to be simply forced or pushed through a tissue lumen can lead to sudden and/or uncontrolled advances into unwanted tissues near the target and or lumen. In addition, particularly directed to navigating the various lumens of the lung, known guidewires may be stiffer than the access devices or catheters they are forced or pushed through to penetrate the lumen. A stiff guidewire may cause a straightening of the access device or catheter during advancement and after alignment within the tissue has been established. This straightening not only may cause a misalignment with the target but may also account for the guidewire to advance at an oblique angle preventing proper tissue puncture or a glancing blow of the wire. Thus, there exists a need to provide guidewires which can be more accurately controlled, require less force to advance through the lumen regardless of stiffness, and decrease the likelihood of misalignment during advancement.

### SUMMARY

The present disclosure describes guidewires configured for passage through a natural lumen wall to an off-lumen target tissue or lesion. The guidewires are further configured to be rotated without causing the guidewire from becoming misaligned.

The guidewires described herein includes a control handle operably coupled to an elongate wire body. The elongate wire body extends between a proximal end portion and a distal end portion. The distal end portion has a distal tip extending distally therefrom. The distal tip may include an atraumatic or traumatic distal tip. An atraumatic distal tip may be a generally rounded smooth tip. A traumatic distal tip may include one or more cutting elements. The control handle is positioned on the proximal end portion of the elongate wire body. The control handle is configured to rotate the elongate wire body and the distal tip about a longitudinal axis thereof.

Methods for deploying the guidewires to an off-lumen lesion are also provided. One method includes positioning a guidewire through a lumen of a patient to a selected exit point in a wall of the lumen near the off-lumen lesion, the guidewire configured to be rotated and including a distal tip, rotating the guidewire and the distal tip, via the handle operably coupled thereto outside the patient, through the wall of the lumen; and creating a path beyond the lumen to the off-lumen lesion by rotating and advancing the guidewire outside the lumen and in a direction towards the lesion. The distal tip may be atraumatic or traumatic.

### BRIEF DESCRIPTION OF THE DRAWINGS

Various aspects and features of the present disclosure are described herein below with reference to the drawings, wherein:

FIG. 1A depicts a side view of a guide wire as described in at least one embodiment herein;

FIG. 1B depicts a schematic perspective view of a handle portion of the guidewire of FIG. 1A as described in at least one embodiment herein;

FIGS. 2A and 2B depict a side and top view, respectively, of a distal tip of a guidewire as described in at least one embodiment herein;

FIGS. 3A and 3B depict a side and top view, respectively, of a distal tip of a guidewire as described in at least one embodiment herein;

FIGS. 4A, 413, and 4C depict a side view, top view, and cross-sectional top view, respectively, of a distal tip of a guidewire as described in at least one embodiment herein;

FIGS. 5A, 5B, and 5C depict a side view, top view, and cross-sectional top view, respectively, of a distal tip of a guidewire as described in at least one embodiment herein;

FIGS. 6A and 6B depict a side view and a top view, respectively, of a distal tip of a guidewire as described in at least one embodiment herein;

FIG. 7 depicts a side view of a distal tip of a guidewire as described in at least one embodiment herein;

FIGS. 8A and 8B each depict a side view of a distal tip of a guidewire as described in at least one embodiment herein;

FIG. 9 depicts a schematic perspective, view of a handle portion of a guidewire as described in at least one embodiment herein;

FIG. 10A is an illustration of an endoscope, assembly including a guidewire inserted into a lung as described in at least one embodiment herein; and

FIGS. 1013 and 10C are enlarged detail views of the circled area of FIG. 10A.

### DETAILED DESCRIPTION

Detailed embodiments of the present disclosure are disclosed herein; however, the disclosed embodiments are merely examples of the disclosure, which may be embodied in various forms. Therefore, specific structural and functional details disclosed herein are not to be interpreted as limiting, but merely as a basis for the claims and as a representative basis for teaching one skilled in the all to variously employ the present disclosure in virtually any appropriately detailed structure.

Aspects of the present disclosure are described in detail with reference to the drawing figures wherein like reference minerals identify similar or identical elements. As used herein, the term “distal” refers to the portion that is being described which is further from a user, while the term “proximal” refers to the portion that is being; described which is closer to a user.

The present disclosure is directed in part to a guidewire configured for improved off-lumen deployment via rotation. The guidewire includes at least a handle portion and a body portion, at least the body portion configured to rotate. The body portion is configured for creating an off-lumen pathway to a target tissue, e.g., lesion. The pathway being subsequently used for the passage of one or more catheters and/or surgical instruments to treat, image, and/or biopsy the target tissue.

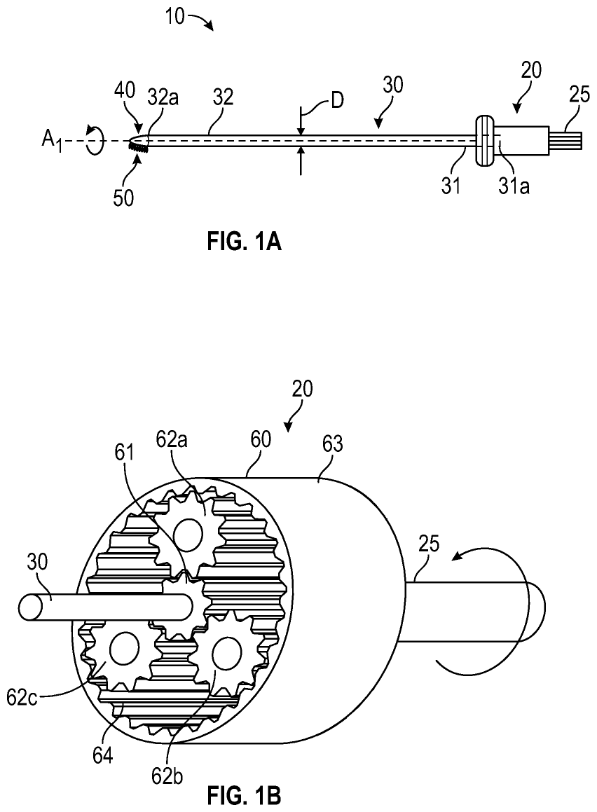

FIG. 1A depicts a guidewire 10 as provided herein. The guidewire 10 includes a, control handle 20 (e.g., handle portion) operably coupled to an elongate wire body 30 (e.g., body portion), The elongate wire body 30 extends between a proximal end portion 31 and a distal end portion 32. The proximal end portion 31, and/or particularly the proximal-most portion 31a, is secured to a portion of the control handle 20. The distal end portion 32, and/or particularly the distal-most end 32a, has a distal tip 40 extending distally therefrom. The distal tip including at least one cutting element 50.

The control handle 20 is configured to rotate the entire wire body 10, including the distal tip 40, about its longitudinal axis A₁ (as indicated by the arrow). The control handle 20 may be manually-operated or power operated. The control handle 20 may be directly operably coupled to the wire body 30 or indirectly operably coupled to the wire body via a coupling device.

As shown in FIG. 18, in some embodiments, the control handle 20 includes at least a rotatable grip 25 operably coupled to one or more gears 60, including but to limited to a planetary gear. A planetary gear 60 includes a central sun gear 61 surrounded by a plurality of planetary gears 62a-c held on a carrier 63 and enclosed within an outer gear ring 64. By rotating the grip 25 (see arrow), the one or more gears 60 are caused to rotate in a manner which causes the wire body 30 to rotate about its longitudinal axis A L The speed and/or torque associated with the spinning wire body may be controlled by how the wire, the handle and the gear are operably coupled. For example, as shown in FIG. 113, in some embodiments, the wire 30 may be coupled to the central sun gear 61 via either direct attachment or indirect attachment via a shaft coupler, overmold, or press-in coupler. In such embodiments, the control handle 20 may be coupled to the outer gear 64 which is coupled to the central sun gear 61 via planetary gears 62a-c. Rotation of the rotatable grip 25 causes the outer gear 64 to rotate at a given speed, which further causes the other inner gears to rotate at a higher speed and potentially a lower torque than the outer gear 64. An increase in rotational speed and/or reduction in torque may be employed to achieve high relative displacement of the tip in rotation relative to axial advancement without rotation thereby requiring less force on the tip to advance through and/or cut through the tissue. In some embodiments, the speed ratio of the outer gear 64 to the central sun gear 61 may be 1:2, 1:3, 1:5, 1:10, 1:20, 1:50, 1:100 and/or the torque ratio of the outer gear 64 to the central sun gear 61 may be 100:1, 50:1, 20:1, 10:1, 5:1, 3:1, 2:1. One example of such a gear may include a speeder gear box.

In still other embodiments, the wire 30 may be coupled to the outer gear 64 via either direct attachment or indirect attachment via a shaft coupler, overmold, or press-in coupler. In such embodiments, the control handle 20 may be coupled to the central sun gear 61 which is coupled to the outer gear 64 via planetary gears 62a-c. Rotation of the rotatable grip 25 causes the central gear 61 to rotate at a given speed, which further causes the other outer gears to rotate at a lower speed and potentially a higher torque than the central sun gear 61. In some embodiments, the speed ratio of the central sun gear 61 to the outer gear 64 may be 100:1, 50:1, 20:1, 10:1, 5:1, 3:1, 2:1 and/or the torque ratio of the central sun gear 61 to the outer gear 61 may be 1:2, 1:3, 1:5, 1:10, 1:20, 1:50, 1:100.

The wire body generally includes one or more elongated core wires. In some embodiments, the wire body includes only one elongated core wire. In some embodiments, the wire body may include a combination of multiple core wires to form the body. The one or more core wires may be made from any combination of suitable biocompatible materials. Some non-limiting examples include metals, such as stainless steel, gold, silver, platinum, tungsten, as well as shape memory materials such as nitinol. The wire body may be a solid wire body or may include a hollow center channel.

As further depicted in FIG. 1A, the wire body 30 may maintain a constant diameter D (or thickness for non-circular embodiments) along the entire length of the wire body 30, not necessarily including the distal tip 40 and/or any proximal portion of the wire body 31a directly coupled to the handle portion 20. The diameter D may range from about 0.02 to about 0.04 inches, and/or particularly, in some embodiments from about 0.025 to about 0.035 inches. Such a wire body may further be configured to display a constant stiffness along the entire length of the body, again not necessarily including the distal tip and/or any proximal portion directly coupled to the handle portion. In some embodiments, however, it is envisioned that the wire body may include at least one tapered section, such as along the distal end portion.

In addition to the one or more core wires, the wire body described herein may further include a helical coil spring wrapped about the outer surface of the core wire along a length of the core wire, and/or particularly from the proximal end portion to distal end portion of the elongate wire body. The diameter of the helical may also remain constant along a length of the wire body. The helical coil may be made from any combination of suitable biocompatible materials. Some non-limiting examples include metals, such as stainless steel, gold, silver, platinum, tungsten, as well as shape memory materials such as nitinol. In some embodiments, the core wire and the helical coil may be made from the same type of material. In some embodiments, the core wire and the helical coil may be made of different materials.

The wire body may define a length ranging from about 10 cm to about 500 cm. In some embodiments, the length of the wire body may be from about 20 cm to about 400 cm. In still other embodiments, the length of the wire body may be from about 50 cm to about 300 cm. In still yet other embodiments, the length of the wire body may be from about 125 cm to about 250 cm.

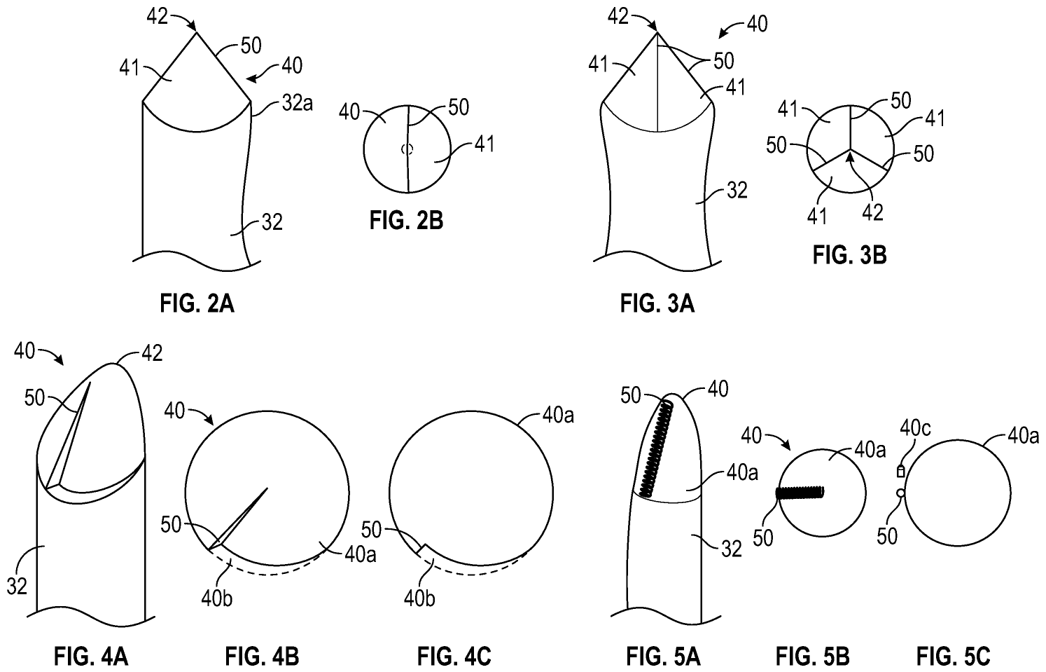

FIGS. 2A-6 depict the distal end portion 32, and particularly the distal-most end 32a, of the wire body 30 anchor the distal traumatic tip 40 including one or more cutting elements 50. For, example, as shown in FIGS. 2A-3B, in some embodiments, the distal tip 40 includes at least two generally conical-shaped portions 41 that each taper to a common distal-most point 42. The one or more cutting elements 50, in such embodiments, may include two or more facets which form cutting edges along the border where the conical-shaped portions 41 meet. In some embodiments, the tip 40 includes two facets (FIGS. 2A-2B). In some embodiments, the tip 40 includes three facets (FIGS. 3A-3B).

The distal tip 40 and the elongate wire body may be connected to each other using any suitable method. In some embodiments, the distal tip and the elongate wire body are a monolithic structure. In some embodiments, the distal tip and the elongate body are separate parts connected via molding, pressing, interlocking, fastening, crimping, and the like.

In some embodiments, as shown in FIGS. 4A-4C, the one or more cutting elements 50 may include a negative cutting feature which is defined in a portion of an outer surface 40a of the tip 40 creating a reduced or removed portion 40b of the outer surface 40a, The negative cutting feature may be formed in the tip using any suitable method including but not limited to grinding, etching, lasering, ultrasonics, and the like. As depicted, a portion 40b is removed from the general contour of the outer surface 40a of the tip 40 to create the negative cutting feature, e.g., flute, extending along any length of the tip 40.

In some embodiments, as shown in FIGS. 5A-5C, the one or more cutting elements 50 may include a positive cutting feature which adds a portion 40c to the outer surface 40a of the tip 40. As depicted, in some embodiments, a weld bead may be added along, the outer surface 40a to create a positive cutting feature distinct from the general contour of the outer surface 40a. The cutting element including a positive cutting feature, e.g., weld bead, may extend along, any length of the tip 40.

The tips described herein may include any number of the cutting elements described herein, individually or in combination. For example, in some embodiments, the tip may include a first cutting element including a positive cutting feature and a second cutting element including a negative cutting feature.

Also, the cutting elements described herein may extend along any length of the tip. For example, in some embodiments, the one or more cutting elements may extend along a majority, if not the entire length, of the tip.

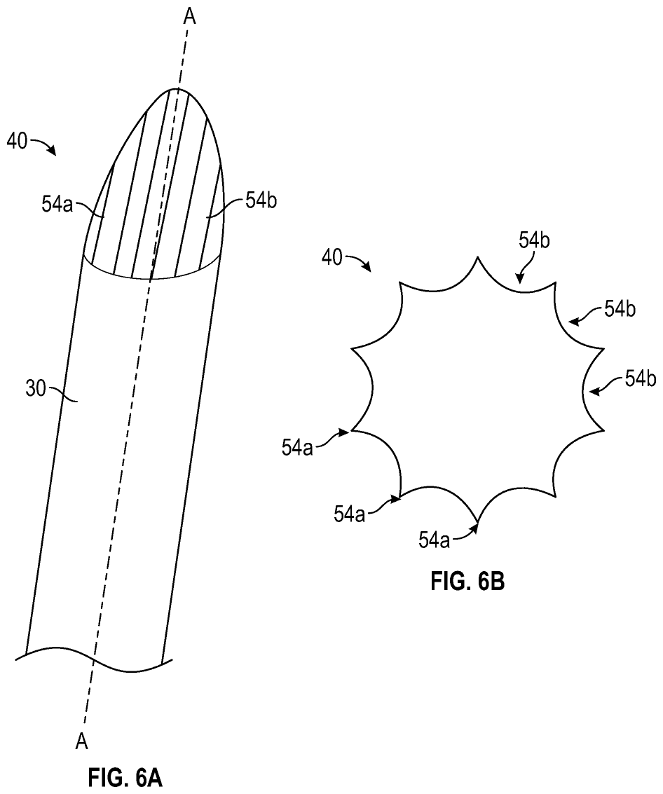

As shown in FIGS. 6A-6B, in some embodiments, the one or more cutting elements 50 may extend only along the longitudinal axis A₁ of the wire body 30. The cutting elements 50 in FIG. 6 include a plurality of striations defining alternating ridges 54a and valleys 54b. The striations may be formed using any suitable method including but not limited to grinding, etching, lasering, ultrasonics, and the like.

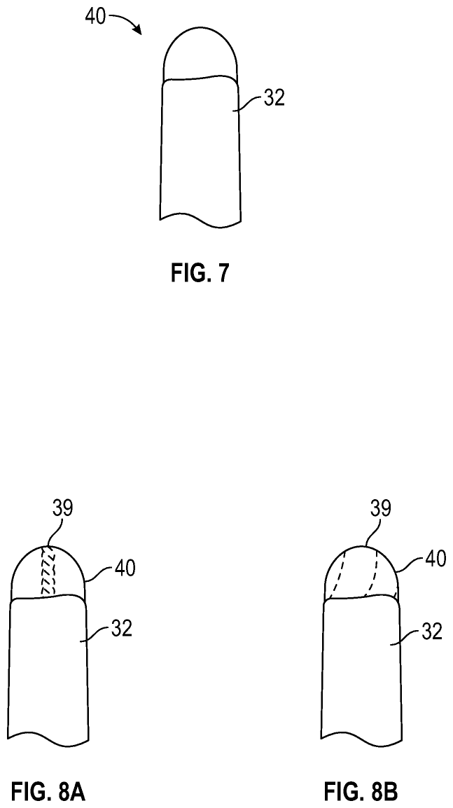

As shown in FIG. 7, in some embodiments, the distal tip 40 may be atraumatic including a generally rounded smooth outer surface. Although atraumatic, the distal tip 40 is still able to penetrate through a tissue wall of a lumen because of the additional rotation of the distal tip 40 as described herein.

As shown in FIGS. 8A and 8B, in some embodiments, the atraumatic distal tip 40 may maintain a generally rounded outer surface further including a non-smooth texture 39. The texture may be added using any suitable method including polishing, grinding, pressing, etching, and the like. In addition, any suitable pattern may be used. For example, as shown in FIG. 8A, the texture 39 may be positioned along a central longitudinal axis of the distal tip 40. In another example, as shown in FIG. 8B, the texture 39 may define one or inure spiral configurations around the distal tip 40.

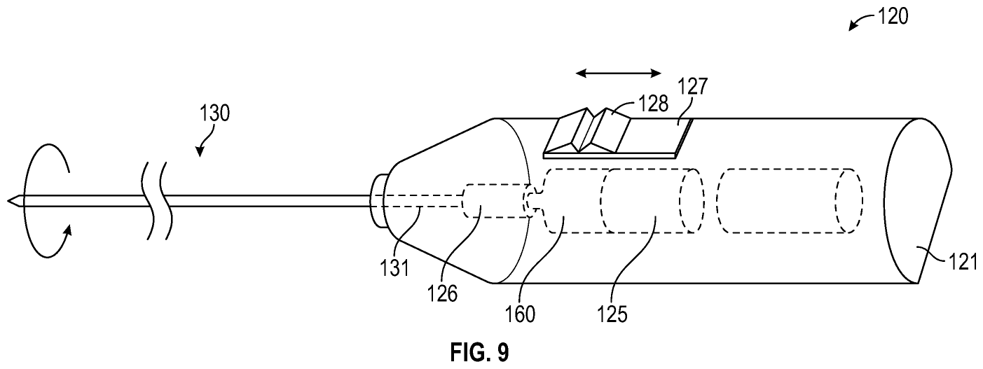

As shown in FIG. 9, in some embodiments, the handle 120 may be a power-operated control handle configured to rotate the guidewire 130. Control handle 120 includes a housing 121 configured to secure at least a motor 125 therein. The motor 125 operably coupled to the proximal end portion 131 of the wire body 130 either directly or via a coupler 126. In some embodiments, the motor 125 may be operably coupled to a planetary gear 160 which is operably coupled to the proximal end portion of the wire body 131.

As further shown in FIG. 9, the housing 121 may define at least one exterior channel 127 sized and dimensioned to receive actuator 128 configured to slide within the channel 127 to control and/or change the speed at which the wire is rotated. In some embodiments, a second actuator (not shown) may be configured to further control the amount of torque applied to the wire body or steer the distal end portion and/or tip of the guidewire.

The guide wires described herein may be formed using any suitable method and/or any suitable biocompatible material known to those of ordinary skill. Some non-limiting examples of methods of forming the catheter, and particularly at least the tube portion of the catheter, include extrusion, molding, casting, pressing, and the like.

The guidewires described herein may be utilized to reach a desired target tissue and/or lesion located off-lumen. By the tissue or lesion being located off-lumen, the guidewire may be designed to: navigate through a given lumen to a given point near the target tissue or lesion; exit the lumen via an exit point created in the lumen wall by rotating the guidewire through the lumen wall; and creating a path beyond the lumen to the target or lesion by continuing to rotate the guidewire in the direction of the target or lesion.

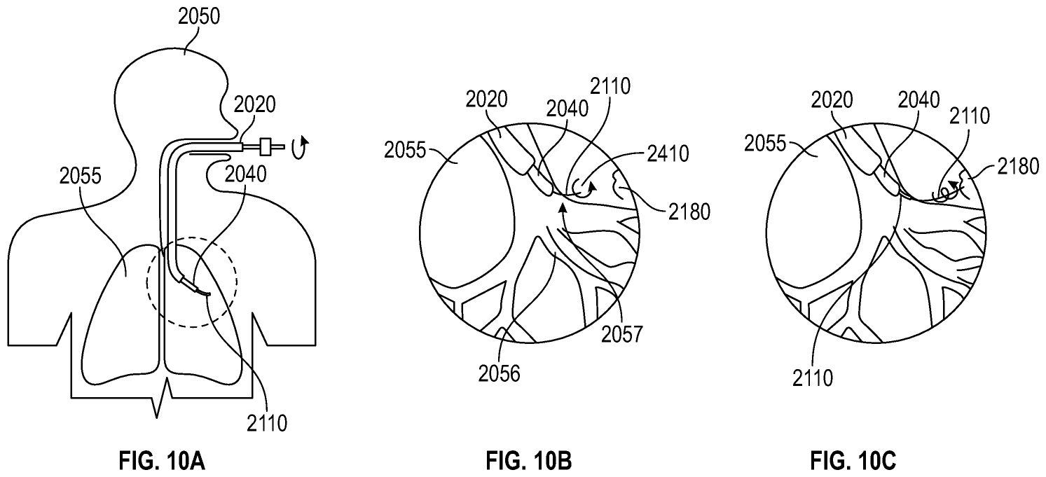

In some embodiments, a method for guidewire deployment to an off-lumen lesion includes: positioning and/or navigating a guidewire as described herein through a lumen of a patient, and particularly a lumen in the lungs, to a selected exit point in a wall of the lumen near the off-lumen lesion (FIG. 10A); rotating the guidewire, via the handle operably coupled thereto outside the patient, and distal tip through the wall of the lumen (FIG. 10B), and creating a path beyond the lumen to the off-lumen lesion by rotating and/or advancing the guidewire outside the lumen and in a direction towards the lesion (FIG. 10C). Withdrawal of the guidewire may also occur.

In some embodiments, the rotation of the guidewire may be manually performed. In some embodiments, the rotation of the guidewire may be automatically performed by a power-operated handle or device.

FIG. 10A illustrates a bronchoscope 2020, a catheter 2040 and a guidewire 2110 as described herein inserted into the lungs 2050 via a natural orifice (e.g., the mouth) of a patient 2150 toward an off-lumen target or lesion 2180 following a pathway plan. The proximal ends, i.e., handles, of the bronchoscope 2020 and catheter 2040 are not shown for clarity purposes. When the bronchoscope 2020 reaches a certain location of the lung 2055, the bronchoscope 2020 becomes wedged and cannot go further into bronchial tree clue to the size constraints. Then, the catheter 2040, with or without an extended working channel, may be used to navigate the luminal network towards an off-lumen target 2180, In some embodiments, the endoscope 2020, the catheter 2040 and/or the guidewire 2110 may follow a predetermined pathway plan associated and/developed using any known lung navigation software, e.g., the ILOGIC® planning suite software currently sold by Covidien LP.

FIG. 1013 illustrates an enlarged detail view of the circled area of FIG. 10A, where the guidewire 2110, and particularly the distal tip 2140 of the guidewire (including any combination of cutting elements described herein), is rotated (see arrows) via the handle 2120 operably coupled thereto, through the wall 2057 of the lumen 2056 exiting the natural lumen 2056 of the lung 2055. Since the speed and/or torque of the rotation of the wire 2110 may be controlled by the handles 2120, less force or push is required to advance the wire 2110 through the lumen wall 2057. In addition, the stiffness of the wire may remain stiffer than the catheter 2040 without causing the catheter to straighten or shift in position to become misaligned.

In some embodiments, the wire and/or the distal tip may be rotated at a speed ranging from about 25 to about 3500 rpms. In some other embodiments, the wire and/or the distal tip may be rotated at a speed ranging from about 50 to about 1250 rpms. In still other embodiments, the wire and/or the distal tip may be rotated at a speed ranging from about 100 to about 3000 rpm.

FIG. 10C illustrates the guidewire 2110, and particularly the distal tip 2140 of the guidewire 2110, continuing to be rotated while advancing (see arrows) creating an off-lumen path beyond the wall 2057 and/or exit point of the lumen 2056 and in the direction of the off-lumen lesion 2180. The path being created by the rotating and/or advancing the guidewire 2110 outside of the lumen 2056 and in a direction towards the lesion 2180. The guidewire 2110 ultimately butting up against the target tissue or lesion 2180. At which point, the catheter 2040 may be extended over the guidewire 2110 to maintain the patency of the path and/or the guidewire 2110 may be withdrawn and replaced with any suitable surgical instrument to treat, resect, and/or biopsy the off-lumen tissue or lesion 2180.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting but merely as exemplifications of particular embodiments. Those skilled in the art will envision other modifications within the scope and spirit of the claims appended hereto.

What is claimed is:

1. A guidewire comprising:
an elongate wire body extending between a proximal end portion and a distal end portion, the distal end portion having a traumatic distal tip extending distally therefrom, the traumatic distal tip including one or more cutting elements, and
a control handle positioned on the proximal end portion of the elongate wire body, the control handle configured to rotate the elongate wire body and the distal tip about a longitudinal axis thereof.

2. The guidewire of claim 1, wherein the one or more cutting elements include two or more facets.

3. The guidewire of claim 2, wherein the one or more cutting elements include three facets.

4. The guidewire of claim 1, wherein the one or more cutting elements include one or more striations extending across at least a portion of the distal tip and aligned with the longitudinal axis of the elongate wire body.

5. The guidewire of claim 1, wherein the one or more cutting elements includes a negative cutting feature defined within a portion of an outer surface of the distal tip.

6. The guidewire of claim 5, wherein the negative cutting feature includes a flute.

7. The guidewire of claim 1, wherein the one or more cutting elements includes a positive cutting feature extending away from a portion of an outer surface of the tip.

8. The guidewire of claim 7, wherein the positive cutting feature includes a weld bead.

9. The guidewire of claim 1, wherein the elongate wire body is made from a single wire.

10. The guidewire of claim 1, wherein the elongate wire body maintains a constant diameter along an entire length of the body.

11. The guidewire of claim 1, wherein the wire body defines a diameter ranging from about 0.02 to about 0.04 inches.

12. The guidewire of claim 1, wherein wire body defines a constant stiffness along an entire length of the body.

13. The guidewire of claim 1, wherein the elongate wire body defines a length ranging from about 10 cm to about 500 cm.

14. The guidewire of claim 1, wherein the handle is manually-operated.

15. The guidewire of claim 15, wherein the handle includes a planetary gear.

16. The guidewire of claim 1, wherein the handle is power-operated.

17. The guidewire of claim 1, wherein the power-operated handle includes at least a motor configured to rotate the elongate wire body, a coupler connecting the proximal end portion of the elongate wire body to the motor, a power source, and a slidable switch configured to control at least the speed of rotation of the elongate wire body.

18. A guidewire comprising:
an elongate wire body extending between a proximal end portion and a distal end portion, the distal end portion having an atraumatic distal tip extending distally therefrom, and
a control handle positioned on the proximal end portion of the elongate wire body, the control handle configured to rotate the elongate wire body and the atraumatic distal tip about a longitudinal axis thereof.

19. A method for guidewire deployment to an off-lumen lesion comprising:
positioning a guidewire through a lumen of a patient to a selected exit point in a wall of the lumen near the off-lumen lesion, the guidewire configured to be rotated and including a traumatic distal tip;
rotating the guidewire and the traumatic distal tip, via the handle operably coupled thereto outside the patient, through the wall of the lumen, and
creating a path beyond the lumen to the off-lumen lesion by rotating and advancing the guidewire outside the lumen and in a direction towards the lesion.

20. The method of claim 19, wherein rotating the guidewire and the distal tip includes rotating at a speed ranging from about 100 to about 3000 rpms.
